(19) United States
(12) Patent Application Publication
(10) Pub. No.: US 2011/0218618 A1
(43) Pub. Date: Sep. 8, 2011

(54) Self-Regenerating Drug-Delivering Stent

(76) Inventor: Stephen H. Monroe, Portland, OR (US)

(21) Appl. No.: 13/039,487

(22) Filed: Mar. 3, 2011

Related U.S. Application Data
(60) Provisional application No. 61/339,394, filed on Mar. 4, 2010.

(51) Int. Cl. A61F 2/82; C08J 5/18; B32B 37/16

(52) U.S. Cl. 623/1.39; 623/1.42; 264/331.11; 156/242

(57) ABSTRACT

A method for bulk manufacture of a microporous sheet material from polyolefin fibers, where an average pore size in the material is controlled by calendering the material through low-energy rollers at a suitable temperature, pressure and speed. The material is useful to selectively pass or reject certain substances, and may be combined in a resilient, tube-shaped configuration to form a self-regenerating drug-delivering vascular stent that reduces inflammation at the stent site to prevent restenosis.

### PRIORITY CLAIM

This application claims the benefit of U.S. Provisional Application No. 61/339,394, filed Mar. 4, 2010.

### FIELD

This invention relates generally to medical devices that are used for treating vascular conditions. More specifically, this invention relates to a novel type of drug-scavenging vascular stent that is capable of self-recharging an active pharmaceutical ingredient either by close or continuously.

### BACKGROUND

Vascular stents have been in use for a number of years as an improved treatment for vascular thrombosis, arthrosclerosis, and other blockages or narrowing of vascular lumens; stents are often preferred to balloon angioplasty. However, insertion of a stent elicits a wound reaction that can cause undesirable effects such as inflammation, infection, thrombosis or proliferation and adhesion of cell growths that can block the vessel and result in restenosis.

Improved stents have been developed to counteract these adverse effects. One important improvement is the drug-eluting stent. These devices are typically coated or impregnated with anti-inflammatory (or other) drugs, which elute over a period of time ranging from clays to months. However, the drugs eventually run out, which can result in a resurgence of the undesirable reaction, leading to restenosis and the need for further surgical interventions.

It would be beneficial to have a stent that could continuously elute an anti-inflammatory or other class of drug, without running out after a period of time. The development of such a drug-eluting stent that could recharge itself with a drug or active pharmaceutical ingredient (“API”) would represent a significant advancement of the technology.

### SUMMARY

The present invention addresses this goal through a novel design that allows the stent to be recharged with an API so that the drug-eluting nature of the stent is regenerated. Embodiments of the invention use a substrate material which is combined with or coated upon a prior-art stent (or the material may also be used to construct the stent substrate itself). The substrate material captures the API either chemically or physically as it travels through the bloodstream, concentrating the API at the stent site. The inventive stent does not, strictly speaking, elute the drug, but rather waylays it or retards its passage in the bloodstream past the stent, resulting in an increased concentration of the API at the stent site. This increased concentration offers similar therapeutic effects to prior-art drug-eluting stents, but the new stent does not rely on a coating that becomes depleted. Instead, if the API is present in the bloodstream (a state that can be easily and non-invasively established, if it is not inherently so) it can be attracted to and concentrated near the stent site, where it can be most beneficial.

### BRIEF DESCRIPTION OF DRAWINGS

Embodiments of the invention are illustrated by way of example and not by way of limitation in the figures of the accompanying drawings in which like references indicate similar elements. It should be noted that references to “an” or “one” embodiment in this disclosure are not necessarily to the same embodiment, and such references mean “at least one.”

FIG. 1 shows a general view of a stent according to an embodiment of the invention.

FIG. 2 shows photomicrographs of a sheet material produced according to an embodiment of the invention.

FIG. 3 outlines a method of producing and using a sheet material according to an embodiment of the invention.

### DETAILED DESCRIPTION

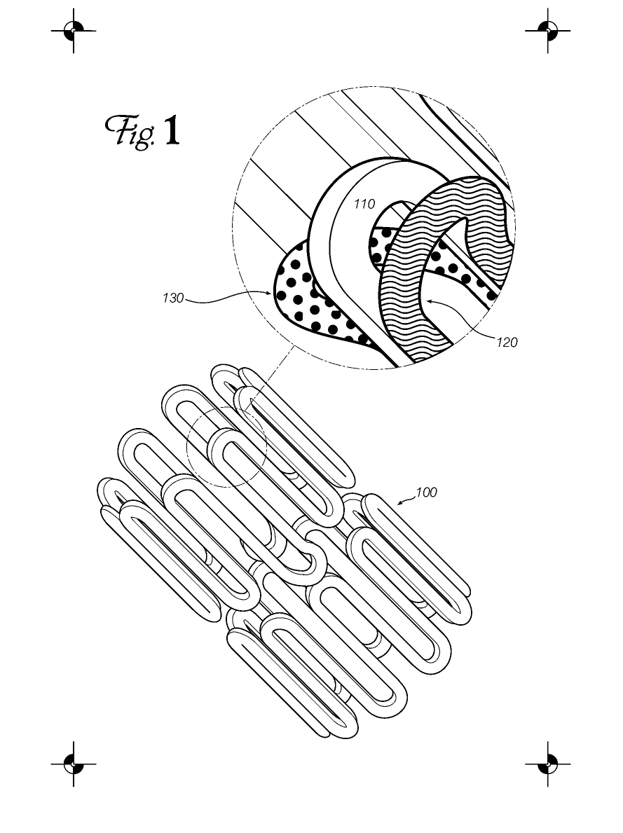

The present specification describes the manufacture of a microporous material in which the pore size is controlled to selectively allow the penetration of individual ions up to a size of approximately 10 Angstroms, and the use of such a material in the construction of a vascular stent. FIG. 1 shows a general perspective view of a stent 100. As is conventional in the art, a stent is a roughly cylindrical or tubular device, constructed so that its outer diameter can be reduced by placing the stent under tension, torsion or compression, or simply by constraining the stent within a smaller tube. The diameter-reduced stent is inserted via catheter to its desired location and then released to return to its original diameter, thus supporting and dilating a narrowed blood vessel.

The stent shown in FIG. 1, element 100 comprises a conventional substrate 110 of a material such as gold, titanium, stainless steel or plastic, with a thin layer of the microporous material 120 adhered thereto. Layer 120 may be laser-cut from a bulk sheet of the material produced as described below. A stent may comprise a second layer of microporous material 130 adhered to the inner surface of the substrate. Alternatively, a stent may be constructed entirely of the microporous material by winding or rolling several layers of the material onto a cylindrical form, then laser-cutting the desired compressible structure (e.g., a serpentine pattern like substrate 110) from the rolled tube of microporous material.

The microporous material improves the function of the stent by filtering specific ions from the bloodstream without causing a buildup of larger molecules and/or cells that could potentially result in accumulations or blockages. The filtering of the bloodstream around the stent results in an increased concentration of certain ions near the stent. More specifically, potassium and rubidium ions are selectively scavenged from the bloodstream. A number of researchers have reported the anti-inflammatory effects of potassium and rubidium ions (see, e.g., Potassium Humate Reduces Inflammation and Clinically Improves the Outcomes of Patients with Osteoarthritis of the Knee by Van Rensberg et al., The Open Conference Proceedings Journal 2010 v.1 pp. 69-74; 2007; Renal inflammation is modulated by potassium in chronic kidney disease: possible role of Smad7 by Wang et al., Am J Physiol Renal Physiol 293:F1123-F1130, 2007). Embodiments of this invention capitalize on these effects and on the abundant availability of these ions in the bloodstream.

By utilizing specific pore sizes in the stent, the present invention allows easy access to the stent media by the target ions, while denying access to larger molecules. The relatively large size of the pores also allows easy passage of other, smaller ions, while slowing the passage of K⁺ and Rb⁺ specifically, resulting in a temporary increase in localized concentration of those ions. This in turn results in the anti-inflammatory effect in the immediate area surrounding the stent as these key ions are scavenged from the bloodstream.

The filtering of these ions is an ongoing process resulting in a continual regeneration of the active ingredients in the stent. Additional actives could be provided to the stent by a bolus injection of a saline solution containing the desired alkali elements as salts.

The stent itself can be made of a number of materials. It can be comprised of a non-woven material treated to provide the desired pore size distribution, or it could be made out of other materials where the pores are artificially created (for example, by sintering powders or by laser etching or drilling). These materials could consist of metals such as gold, platinum, steel or other or they could be made of plastics such as high molecular weight polyethylene or other thermoplastics, or they could be made of ceramics or any other material in which pore size can be induced and controlled.

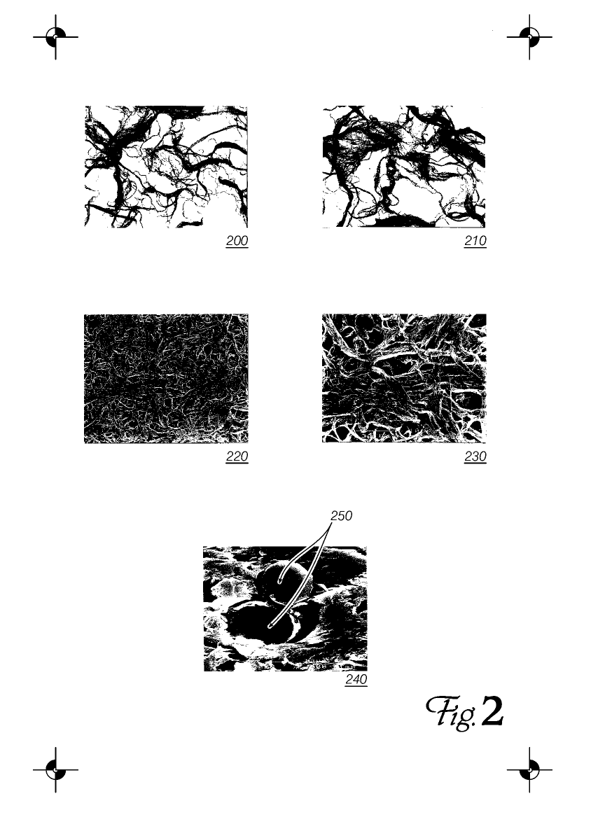

In a preferred embodiment, the inventive stent comprises a plastic-fiber-based “paper.” This paper is made in a bulk process that is similar to the manufacture of ordinary wood-pulp-based paper. The plastic paper is made from synthetic polyolefin pulp fibers, which may consist primarily of polyethylene fibers or polypropylene fibers or a mixture of each. Additional fiber can be added to the paper to achieve specific characteristics such as binding, wet tensile strength, spring strength, plastic memory, surface energy or other features. FIG. 2 shows several photomicrographs of raw materials, intermediate processing products, and a finished polyolefin pulp sheet. The pulp fibers (e.g., 200 and 210) are made of polyethylene fibers that have been flash spun in order to produce a structure much like highly-defibrillated wood pulp. This allows the fibers to dramatically increase their surface binding area. It also permits the controlled compression of the resulting substrate. This in turn permits the structure to be designed to have a specific pore size distribution. Suitable pulp fibers are available commercially from Mitsui Petrochemical Industries, Ltd. of Tokyo, Japan under the trade name Fybrel® E-9400.

After traditional paper-like processing (described below), the substrate consists of interconnected pores throughout the entire three dimensions of the substrate (see 220, 230). The pores are too large for the purposes herein proposed and must be significantly reduced. (Before reduction, the pores may be large enough to trap large debris, cells, and any other smaller material.)

Pressing or calendering the substrate collapses and effectively shrinks these pores by allowing the synthetic fibrils to flow. The pore size of the sheet can be controlled by the pressure, temperature, residence time in the nip and number of cycles through the calender. The surface energy characteristics of the material can also be manipulated to help achieve the desired results. Photo 240 shows a cross-section of a calendered polyolefin-fiber sheet. In this view, two thicker fibers 250 are also visible. As noted above, such fibers may be added to change secondary characteristics of the bulk sheet product.

In a preferred embodiment of this invention, the sheet is calendered until the average pore size is in the range of 4-10 Angstroms in diameter. At this size, the pores are large enough to allow elemental ions to pass through the substrate, while preventing larger molecules from doing the same. The ultimate goal of the calender processing is to allow the easy passage of elemental alkaline earth metals such as lithium and sodium, while slowing clown the passage of potassium and rubidium. This results in a higher steady-state concentration of the latter elements in the immediate vicinity of the stent. This in turn provides development of a pharmaceutically effective concentration of these cations.

Potassium has been found by many researchers to have a powerful anti-inflammatory effect. Rubidium has been noted to have the same. In addition, the combination of these two provides an environment that helps prevent adhesion of leucocytes, neutrophils and other cell types. It also locally clown-regulates matrix metalloproteinases, which helps the local healing process resulting from the mechanical damage clone by the stent implantation itself.

By filtering or scavenging potassium and rubidium ions from the bloodstream and producing a locally-increased concentration, an embodiment of the invention mimics an anti-inflammatory API-eluting stent to help prevent restenosis. By continually recharging itself with the active ions from the bloodstream, it acts like a renewable drug-eluting stent.

Embodiments of the invention solve a significant problem of prior-art drug-eluting stents, which is that the active ingredient in the stent becomes depleted over time. By doing so, it is believed that this approach will provide the benefits of a drug-eluting stent without the downside. It is also believed that this approach will act as a long-term or perpetual drug-eluting stent to help prevent restenosis. This in turn will help reduce the need for future replacement surgeries often seen with current drug-eluting stents.

The substrate can be manufactured in volume either on papermaking equipment of virtually any scale, or on non-woven equipment. The individual fibers can be handled using either an aqueous system or dry or airlaid or any other conventional paper-making system designed to entangle and produce a mixed fiber substrate.

The preferred manufacturing method uses an aqueous system where the pulp fibers are mixed with an aqueous carrier such as water. The fibers are added individually at an appropriate concentration. The synthetic pulp is mixed at a consistency up to 4% on an oven-dried basis. Longer fibers and binder fibers (if necessary) can be dispersed together at a consistency up to 1% oven-dried basis. One possible stock formulation is:

| | |
|---|---|
| Synthetic Pulp | 87% |
| Staple Fiber | 7.5% (2.2 denier × 10 mm) |
| Binder fiber | 5.5% |

The fibers are mixed in a high-shear system until complete dispersion has taken place. Excessive cutting of the fibers should be avoided in any refining of the stock.

The pulp slurry is laid upon a wire frame as in any normal wood-pulp product and produced like a normal sheet of uncoated paper. Depending upon the machine used, modifications of the drying system may be necessary to achieve the desired properties. Suitable properties for two example embodiments are shown here:

| Sheet Properties | | Example 1 | Example 2 |
|---|---|---|---|
| Basis Weight (lbs/3300 Sq. Ft) | | 48 ± 3 | 48 ± 3 |
| Caliper | | 7.5 ± 0.5 | 5.7 ± 0.5 |
| Sheffield smoothness | | 350 ± 35 | 120 ± 15 |
| Opacity | | 94 ± 1 | 94 ± 1 |
| Gurley Porosity | | 32 ± 12 | 60 ± 15 |
| Gurley Stiffness | MD | 63 ± 14 | 40 ± 12 |
| | CD | 51 ± 13 | 35 ± 9 |
| Tear | MD | 36 ± 2 | 37 ± 4 |
| | CD | 43 ± 5 | 45 ± 4 |
| Tensile | MD | 5.0 ± 0.6 | 5.4 ± 0.7 |
| | CD | 3.8 ± 0.3 | 4.1 ± 0.5 |
| Elongation | MD | 7 ± 2 | 9 ± 2.4 |
| | CD | 8 ± 2.5 | 11 ± 2.5 |

The sheet is then calendered with a low-surface-energy roll such as a Teflon-coated calender at a specific pressure, time and temperature to reduce the average pore size to the desired dimension. The preferred average pore size for some embodiments is around 10 Angstroms.

The pore size can be adjusted as desired by changing the pressure, time, or temperature of the calender set up. The specific parameters will change depending on the calender set up, the roll diameter and the roll hardness. The result will be a consistent sheet-thickness or caliper.

The sheet can then be cut and adhered to a bare metal (or other material) stent as a tube-shaped outer covering over the whole (roughly cylindrical) stent or over only the cross-hatched or serpentine design of the stent substrate (as shown, for example, in FIG. 1). Since the sheet is pliable, it can be compressed along with the stent body during insertion. Alternatively, the cut sheet can be adhered to the inner circumference of the stent (again, either as a full tube-shaped inner covering or as a trimmed cover over the stent material).

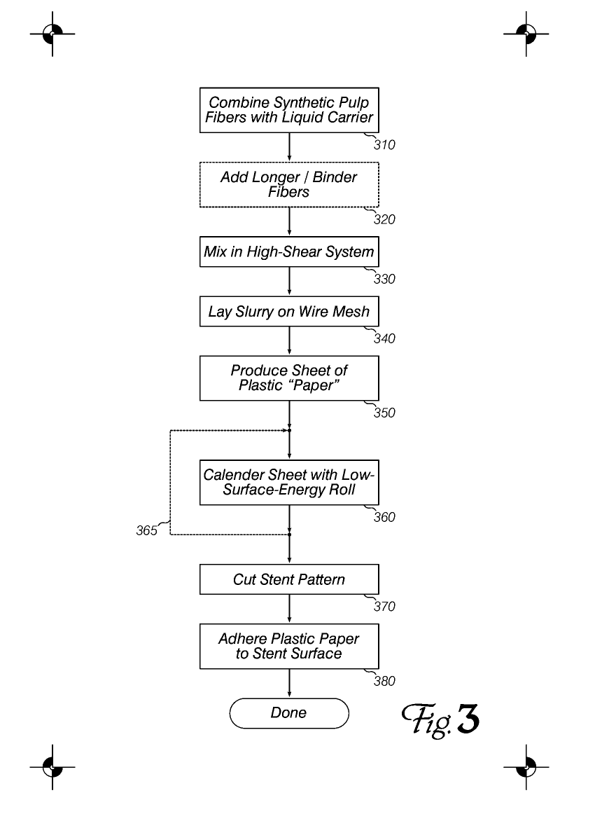

FIG. 3 outlines the production of an embodiment of the invention. First, polyethylene or polypropylene pulp fibers are mixed into a liquid carrier (310). Additional, longer fibers may also be added to obtain specific characteristics of the bulk material (320). Next, the slurry is mixed in a high-shear system until the pulp is well-dispersed and uniform (330). Now, as in ordinary wood-pulp paper manufacture, the slurry is laid out on a wire mesh (340) and dried. When dry, the sheet of plastic “paper” is removed (350) and run through a calender (360) (possibly multiple times 365) to obtain the desired average pore size and other characteristics. Finally, the calendered sheet is cut into an appropriate shape (370) and adhered to the stent substrate (380).

It is preferred that the surface energy of the plastic “paper” material is low so that there will be a reduced tendency for adhesion or attachment by components in the blood stream, including, but not limited to proteins, polysaccharides, macrophages, T-cells, neutrophils, or other types of molecules or cells.

The small pores in the microporous sheet will deny entrance to anything but atomic- or ionic-sized elements. Larger molecules and cells will pass unimpeded through the stent channel. The atomic-sized elements will enter the pores and be filtered from the bloodstream. The very small elements such as lithium, sodium, zinc, magnesium, chlorine and others will pass through the sheet relatively easily, maintaining the same steady-state concentration as the blood plasma. The larger atoms such as potassium, rubidium, and others like them, with ionic diameters closer to the 10 Angstrom average pore size, will pass through the sheet more slowly and effectively reach a higher relative steady-state concentration compared to the blood plasma.

The increased concentration of potassium and rubidium ions in the vicinity of the stent will produce an anti-inflammatory effect, mimicking the effect of an anti-inflammatory drug-eluting stent and helping to slow or prevent restenosis at the stent site. This effect will be longer-lived (potentially permanent) compared to a drug-eluting stent because the potassium and rubidium ions will be continuously replenished as these elements are captured from the blood plasma.

In another group of embodiments, the stent substrate may be made of other materials such as plastic films or thin metals such as gold, titanium, or other materials, wherein pores have been formed with an appropriate average size. As in other embodiments, the preferred average pore size is 10 Angstroms, but sizes ranging up to about 40 Angstroms may also find therapeutic applications.

The embodiments of the present invention have been described largely by reference to specific examples. However, those of skill in the art will recognize that self-regenerating drug-eluting stents, or stents that mimic such behavior by passively concentrating drugs captured from the bloodstream in the area of the stent, can also be constructed of other materials produced by bulk paper-manufacturing techniques, as well as materials having the desired average pore size produced by other methods. Such variations and implementations are understood to be captured according to the following claims.

I claim:

1. A method comprising:
combining polyolefin pulp fibers with a liquid carrier;
mixing the polyolefin pulp fibers and liquid carrier in a high-shear system to produce a uniform slurry;
laying the uniform slurry on a wire frame;
drying the uniform slurry to produce a sheet product; and
calendering the sheet product at least once to reduce an average pore size of the sheet product.

2. The method of claim 1 wherein the polyolefin pulp fibers are at least one of polyethylene pulp fibers or polypropylene pulp fibers.

3. The method of claim 1 wherein the polyolefin pulp fibers are Fybrel® E-9400 fibers.

4. The method of claim 1 wherein the liquid carrier is a water-based carrier.

5. The method of claim 1 wherein the average pore size of the sheet product is reduced to between about 4 Angstroms and about 10 Angstroms.

6. The method of claim 1 wherein the at least one calendering operation is performed with a Teflon-coated calender roll.

7. The method of claim 1, further comprising:
cutting the sheet product to match an outline of a stent; and
adhering the cut sheet product to the stent.

8. The method of claim 1, further comprising:
cutting the sheet product in a shape that can be curled into a tube; and
adhering the sheet product to at least one of an inner circumference of a stent and an outer circumference of a stent.

9. A method comprising:
producing a sheet of paper-like material from a pulp mixture comprising polyolefin pulp fibers;
calendering the sheet of paper-like material using a low-surface-energy calender roll system to make a microporous sheet material having an average pore size between about 4 and about 40 Angstroms; and
incorporating a portion of the microporous sheet material into a vascular stent.

10. The method of claim 9 wherein the producing operation comprises at least one of:
mixing, spreading and drying a uniform aqueous pulp slurry;
pressing a dry pulp mixture; or
utilizing an airlaid nonwoven substrate system; or
utilizing a drylaid nonwoven substrate system.

11. The method of claim 9 wherein the low-surface-energy calender roll system comprises a Teflon-coated calender roll.

12. A vascular stent comprising:
a flexible, substantially cylindrical structure whose outer diameter can be reduced for insertion through a catheter; and
a microporous covering over the cylindrical structure, wherein
the microporous covering has an average pore size between about 4 Angstroms and about 10 Angstroms.

13. The vascular stent of claim 12 wherein the cylindrical structure is formed from at least one of gold, titanium or stainless steel.

14. The vascular stent of claim 12 wherein the cylindrical structure is formed from thermoplastic.

15. The vascular stent of claim 12 wherein the cylindrical structure is formed from a rolled tube of microporous covering material similar to the microporous covering.

16. The vascular stent of claim 12 wherein the microporous covering is formed from a polyolefin-fiber-based, calendered sheet.

17. A self-regenerating drug-delivering stent comprising:
a stent substrate; and
means for scavenging physiologically-active agents from a bloodstream near a site of the stent substrate.

18. The self-regenerating drug-delivering stent of claim 17 wherein the means for scavenging comprises a polyolefin-fiber-based sheet material having an average pore size between around 4 Angstroms and around 10 Angstroms.

19. The self-regenerating drug-delivering stent of claim 17 wherein the means for scavenging comprises a polyolefin-fiber-based sheet material having an average pore size selected to approximate an ionic diameter of a target ion.

20. The self-regenerating drug-delivering stent of claim 17 wherein the means for scavenging is permeable to sodium ions, semi-permeable to potassium and rubidium ions, and substantially impermeable to molecules.

21. The self-regenerating drug-delivering stent of claim 17 wherein the physiologically-active agents are potassium and rubidium ions.

22. The self-regenerating drug-delivering stent of claim 17 wherein the physiologically-active agents suppress a wound reaction at the site of the stent substrate.

23. The self-regenerating drug-delivering stent of claim 17 wherein the physiologically-active agents reduce inflammation near the site of the stent substrate.

24. The self-regenerating drug-delivering stent of claim 17 wherein the physiologically-active agents deter adhesion of molecules and cells near the site of the stent substrate.
